(19) United States
(12) Patent Application Publication
(10) Pub. No.: US 2014/0108086 A1
(43) Pub. Date: Apr. 17, 2014

(54) PROJECT CATEGORIZATION AND ASSESSMENT THROUGH MULTIVARIATE ANALYSIS

(71) Applicant: FLUOR TECHNOLOGIES CORPORATION, Aliso Viejo, CA (US)

(72) Inventor: Robert Prieto, Princeton Junction, NJ (US)

(73) Assignee: FLUOR TECHNOLOGIES CORPORATION, Aliso Viejo, CA (US)

(21) Appl. No.: 14/053,890

(22) Filed: Oct. 15, 2013

Related U.S. Application Data
(60) Provisional application No. 61/713,703, filed on Oct. 15, 2012.

(51) Int. Cl. G06Q 10/06

(52) U.S. Cl. CPC G06Q 10/06313
USPC 705/7.23

(57) ABSTRACT

A system for project categorization and assessment that can employ multivariate analysis techniques to classify a current project by using attributes of the current project to identify project objects representing completed projects similar to the current project. Project data sets of points in a project lifetime can be represented as pictures, having attribute pixels. Pattern recognition techniques can be used on the project pictures. The system can generate eigenprojects for large project object groups or for classification across multiple groups. Aspects of a classified current project can be assessed to suggest project management actions.

This application claims the benefit of priority to U.S. provisional application 61/713,702 filed on Oct. 15, 2012. U.S. provisional application 61/713,702 is incorporated by reference in its entirety.

### FIELD OF THE INVENTION

The field of the invention is project management technologies.

### BACKGROUND

Large scale projects, especially capital construction projects, are notoriously difficult to manage. Project managers, or other stakeholders, require techniques to quickly assess a current state of their project. Ideally, the project managers would be able to quickly compare their project against historical projects or known best practices. Unfortunately, there are very few techniques available to project managers that allow them to recognize the project's state as being similar to circumstances related to other projects.

There are many techniques available to recognize faces or other objects based on multivariate statistical techniques. For example, U.S. Pat. No. 7,907,774 to Parr et al. titled “System, Method, and Apparatus for Generating a Three-Dimensional Representation from one or more Two-Dimensional Images”, filed Jan. 29, 2010, describes using multivariate analysis with respect to facial recognition. It has yet to be appreciated that such techniques could also be applied to project analysis as an aid to project managers.

At best, multivariate analyses have only be used with respect to processing financial project analytics. For example, U.S. patent application 2005/0119959 to Eder titled “Project Optimization System”, filed Dec. 12, 2001, describes using multivariate analysis to identify interrelationships among financial project factors and financial performance.

These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

Interestingly, no known effort has been directed to applying recognition technologies to identify projects or project states as being similar to known types of projects. The Applicant has appreciated that such technologies can be used to aid project managers properly assess their projects as described in the Applicant's work below.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Thus, there is still a need for technologies capable of recognizing projects or project states from attributes of a project, assess the condition of the project based on these attributes, and provide recommendations for the project based on the assessed condition.

### SUMMARY OF THE INVENTION

The inventive subject matter provides apparatus, systems and methods in which one can leverage a project analysis system to recognize a project as being similar to known projects or project types. One aspect of the inventive subject matter includes a project analysis system that includes a project recognition engine.

The project recognition engine can obtain one or more project attributes that describe a project from a project interface. The engine uses the project attributes to identify one or more project objects representing known projects having similar attributes.

The project objects can be created using data sets corresponding to one or more of historical data, performance data, benchmark data, reference data, optimized data, market data, sentiment data, and survey data related to the project represented by each project object. In embodiments, data sets can be constructed composed of data snapshots of a completed project from various points in time. These data snapshots can be thought of as “pictures”, which can provide a basis for an initial group definition of the project. A collection of these pictures from completed projects create a multivariate image over time. The project attribute values making up these pictures can be considered the “pixels” of the picture. In embodiments, multiple multivariate statistical techniques can be employed for the classification of a project picture of a completed project into a defined group. Adding completed, historical or other reference project pictures over time provides additional data with which to adjust the project class. This increases the accuracy of the representation of projects by the project classes and project objects within the classes, and allows for the evolution of reference project objects over time, to account for changes in the projects represented by the project objects that occur over time.

In embodiments, project pictures can also be generated for the current project based on the received project attributes.

In embodiments, project objects for the current project can be generated based on the received project attributes.

To analyze pictures corresponding to a current project, the project objects can be identified through a multivariate analysis, or other algorithm, applied to attributes of the project objects and the project attributes of the current project.

Both in constructing reference project objects and project classifications, and in analyzing current projects against project objects, multiple pictures can be used over time to construct a project “movie” that can illustrate aspects or characteristics as they change and evolve over time during the lifetime of a project.

In embodiments, the project recognition engine can construct an “eigenproject”, which can be constructed from a set of eigenvectors related to a covariance matrix associated with a data matrix made up of project pixels times the number of pictures for a project. The eigenproject can be used to reconstruct a project picture.

Once identified, project objects considered similar to the current projects can be presented via an output device (e.g., project interface, browser, computer, printer, etc.). The project objects can then be used to offer recommendations or suggestions on possible alterations to the current project.

The recommendations can include recommended adjustments to aspects of the current project with the goal of bringing the current project “in line” with the project represented by the project object.

The recommendations can include reclassifying the current project as a project of a different type, such as because project objects of the new type more closely align with the current state of the current project.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic of project recognition ecosystem.

FIG. 2 provides an overview of a sample method of project categorization and assessment, according to an embodiment of the inventive subject matter.

FIG. 3 provides a detailed view of the classification step, according to an embodiment of the inventive subject matter.

### DETAILED DESCRIPTION

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, cellular or other type of packet switched network.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of this document the terms “coupled to” and “coupled with” are also used to mean “communicatively coupled with” where two or more devices are able to communicate over a network.

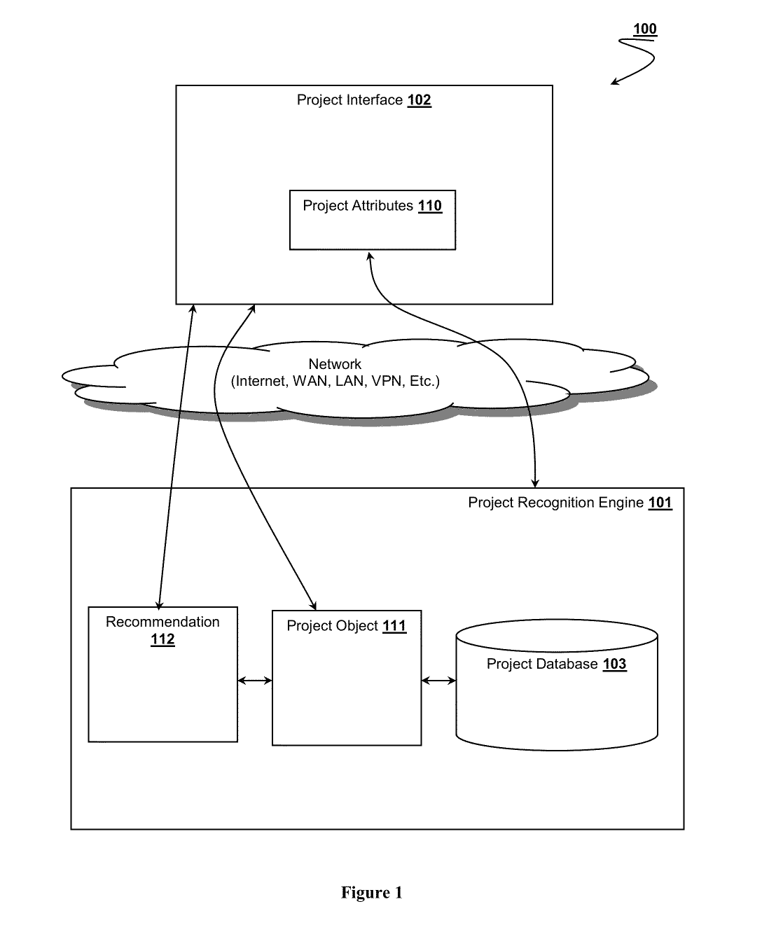

In FIG. 1 project analysis system 100 can include a project interface 102 through which project attributes 110 of a project to be analyzed can be received by the system 100. The project interface 102 can be an interface such as a browser, an application, a computer terminal, an http server, etc., through which a project manager or other stakeholder in the project can submit the project attributes 110. The project interface 102 can include communication interfaces with external data sources (e.g., websites, databases, servers, sensors, equipment, machinery, etc.) from which the system 100 can gather data associated with or corresponding to project attributes 110. An example of a type of project for which the system 100 can be used includes large scale construction projects. Other examples of projects can include financial projects, engineering projects, design projects, construction and construction management projects, software development projects, supply chain projects, maintenance projects, and movie projects. Projects can also be sub-projects of larger, broader projects. For example, one or more of a maintenance project, an electrical project, a structural project, a landscaping project, a zoning project, and a materials project can be a subproject of an overarching construction project. Still further, the projects could represent phases within a project. The network illustrated in FIG. 1 can be a data exchange network such as one of the networks listed above.

Projects can be complex affairs having a wide range of project attributes. The project attributes 110 can be considered to be factors, characteristics, variables, features or parameters associated with a current project. The project attributes 110 can affect, and be reflective of, the state of the project and the progress or execution of the project. One or more project attributes 110 can include attribute values, which can be indicative of a magnitude, amount, percentage, probability, state, condition, stage, etc., of a corresponding project attribute 110.

Examples of project attributes can include a project purpose, a project type, a project goal, a project size, a project execution approach, a project progress, a project challenge, a project location, a project contract, a project contract detail, a project contract obligation, a project risk assessment, a project work load, a perception of an objective (e.g., global perception, current perception, etc.), a resource metric, a stakeholder attribute, a stakeholder number, a degree of support, a prior history, a project driver, a relationship (e.g., a relationship between project team members, a relationship between a project manager and team members, a relationship with a client, a relationship of the project with other projects, a relationship between attributes, etc.), a budget, a sensor reading, a diagnostic attribute, an operational attribute, a cost, an environmental factor, a complexity attribute, a project sentiment attribute, and a project manager attribute.

Project attributes 110 can include one or more of: “global” attributes common to all projects, attributes common to similar projects (e.g., projects of a similar type, of a similar class, projects having similar goals or objectives, similar project aspects, etc.), and attributes specific to a particular project, project type or class (e.g., specific to a particular project type or project class, specific to a particular individual project, specific to a particular aspect of a project, etc.). For example, the global attributes can include project size, budget, location, costs, progress, and other attributes that can be reflective of general, high-level, or otherwise universal project characteristics applicable to all projects. At the other end of the spectrum, a large-scale construction project can include project attributes such as construction code attributes, materials attributes, zoning attributes or other attributes that are specific to construction projects, and would likely not be applicable to projects such as a software development project or a movie project.

Project attributes 110 can be grouped or categorized according to shared commonalities among several attributes, such as attributes common to or contributing to a particular project factor. For example, a group of attributes related specifically to stakeholder aspects of a project can be grouped as stakeholder attributes. These can include a stakeholder identifier, a number, a degree of support, a stakeholder prior history, etc. In some cases, grouped attributes can have established relationships. For example, these relationships can dictate that the inclusion of one of the grouped attributes requires the inclusion of some or all of the remaining grouped attributes, or that a change to a value of a grouped attribute results in a change to one or more of the other grouped attributes according to the rules or parameters of the relationship.

System 100 can further include a project database 103 storing multiple project objects 111, where each project object 111 can represent one or more of a known, previously executed, benchmarked, optimized, reference, or historical project. A project object 111 can represent the entirety of a project, or an aspect or portion of a project (e.g., a division, a task within a project, a sub-project, a project stage, a project state, a project detail, etc.). The project objects 111 can be considered distinct manageable units of project knowledge having object attributes, preferably in the same namespace as the as the current project's attributes. In embodiments, project objects 111 can represent complete projects, project phases, simulated or statistical projects, a project's snap shot in time, project states, project stakeholders, project actions, project trends, project objectives, types of projects, or classes of projects. Project objects 111 can be grouped or categorized. In an embodiment, project objects 111 can be grouped or categorized according to a reference project class.

In an embodiment, project objects 111 can include a project object type referred to as an “eigenproject”, which is discussed in further detail below.

Project objects 111 can represent projects of various levels of success. As such, multiple project objects 111 can exist for a particular project, where one or more of the project objects 111 can represent various successful completions of the project (e.g., optimal completion, ideal completion, minimum acceptable completion, etc.), and where one or more of the project objects 111 can represent unsuccessful completions of a project (e.g., projects that did not reach their goals, projects abandoned or otherwise aborted, etc.). For each of these project outcomes, the project objects can have corresponding object attributes of the project at a particular point in time or other time slices. As these attributes contributed to the project outcome, it enables for assessment of a project and prediction of a degree of success or failure of projects under analysis, and enables a projection of trend data to a measured or pre-determined outcome.

The system 100 further includes a project recognition engine 101 configured to analyze received project attributes with respect to the project object attributes for a project currently under analysis in an attempt to identify project objects that could be considered similar to the current project's state. As such, the project attributes 110 can be considered to represent a snap shot in time or a time slice of the current project. The project recognition engine 101 can further be configured to assess the current project against identified project objects, such as to identify significant attributes of the current project or deviations of the project from the project objects. The project recognition engine 101 can be configured to generate, based on the assessments, a recommendation 112 for presentation to a project manager or other requesting party. In an embodiment, the project recognition engine 101 can be configured to automatically implement some or all of a generated recommendation 112 by causing other computing devices associated with a project to perform adjustments of project parameters associated with project attributes (e.g., calibration of equipment, adjusting operational conditions or parameters, adjust a maintenance schedule, sound an alarm, etc.).

The project recognition engine 101 can comprise computer-readable instructions stored on a non-transitory computer-readable medium that, when executed by a computer processor, carry out functions corresponding to methods and processes of the inventive subject matter. In embodiments, the project recognition engine 101 can comprise a dedicated computing device, having dedicated hardware and/or software that, when executed by the computing device, carry out functions corresponding to methods and processes of the inventive subject matter. In embodiments the project recognition engine 101 can comprise a dedicated hardware processor, specifically configured to execute functions corresponding to methods and processes of the inventive subject matter.

The project recognition engine 101 can be communicatively coupled to the project interface 102, enabling the project recognition engine 101 to receive information (e.g., project attributes 110, other information related to functions and processes of project management) from the project interface 102 and return information (e.g., presentation of identified project objects, recommendations, etc.) to the project interface 102 for display, presentation, and/or implementation.

In an embodiment, the project database 103 can be integral to the project recognition engine 101. In an embodiment, the project database 103 can be communicatively coupled to the project recognition engine 101, whereby the project recognition engine can exchange information with the project database 103 for the purposes of carrying out functions and processes associated with the inventive subject matter. The project database 103 can comprise a non-transitory computer-readable storage medium (e.g., hard drive, server computer, flash drive, optical storage, ROM, etc.) on which project data can be stored.

To present information such as identified project objects, assessments, and/or recommendations, the project interface 102 can include or be communicatively coupled to an output device. Examples of an output device can include a display, audio output devices, a printer, sensory feedback devices, etc.

The system 100 can be employed to assist project managers in understanding, categorizing, assessing and monitoring a project based on consideration of a large number of project attributes 110.

These project attributes 110 can be used to create a pattern definition that can be considered a “picture” of the project. This picture can then be compared with other similar pictures, such as those of existing project objects. The comparison and classification can be performed using pattern recognition techniques utilizing multivariate analysis. Pictures can be similarly grouped and categorized where each category has certain common descriptive features, and can also incorporate anticipated or known project attributes common to particular groups or categories. The project picture for a current project can be retaken over time (e.g., over the project lifetime, potentially including its operating phase) and its strength of correlation with its initially assigned group measured over time, such as to confirm whether the initial assignment was correct.

This pattern recognition approach utilizing multivariate analysis can be used by the system 100 to perform granular categorization of discrete aspects of the project. As an example, a series of pictures of stakeholder relationships taken over time not only allows for early categorization (thus facilitating strategy identification), but also enables for the identification of subtle shifts and trends that might be leading indicators of problems or success. In this respect, statistically meaningful portions of the picture can be compared for characterization or analysis at a desired level of granularity. As a metaphor, this would be similar to, in facial recognition, looking at the eyes of all Caucasian men to further categorize by shape or color.

To create the project objects 111 used to categorize and analyze active projects, data sets initially composed of project pictures from different points in time from completed or historical projects can be initially constructed and provide a basis for initial “group” or “classification” definition. These definitions can be strengthened as additional pictures from newly completed projects are subsequently added. In effect, the initial data set can act as a training set for the developed tool. Thus, these pictures become project objects 111 according to group definitions.

“Pictures” from the same project over time can be stacked to create a multivariate image where time is the third dimension, and each individual image is comprised of rows and columns of “pixels” where columns correspond to similar types or dimensions of project attributes (e.g., client, complexity, environmental factors, stakeholder attributes, etc.) within a normalized attribute namespace. These multivariate images can be used to confirm group classification while individual pictures provide some initial sense of directionality (i.e., classification is getting stronger or weaker). Within a given group, anticipated changes in the multivariate picture over time can be modeled (e.g., after observed behaviors in completed/benchmark/reference projects) and can be employed by the project recognition engine 101 to assess project evolution. Select defined portions of the picture (which can be thought of as features of a project picture, such as the eyes, mouth, ears, nose, etc., recognized and categorized in an image of a face in facial recognition) can be separately characterized to support a increasingly granular analysis, while trading off some of the insights and assessments that can be made from non-obvious or seemingly unrelated correlations.

“Pixels” are considered to be the variables in the analysis and for a given project these variables are expected to be highly correlated. In project pictures, the pixels can be considered to be the attributes associated with a project. These attributes can correspond to the variables routinely tracked as part of a project management system and reported on project status reports. For example, pictures comprised of all attributes (pixels) from a complete project status report at each period through the project lifetime could be used to create the initial project image database and categorization.

The project recognition engine 101 can be configured to “unpack” each image column wise such that sequence of parameters would be the same for each project picture so unpacked. This can enable subsequent analysis of select portions of the data set such as stakeholder data, productivity related data, and external factors assessments.

Each picture's data can be considered as a row of unpacked data in a data matrix. The database of unpacked data is cumulative such that as a new picture is added to the database, effectively one additional row is added. The data matrix can be considered to be equal to the number of pixels times the number of pictures.

As current projects are completed, the pictures from the completed projects can be added to the data used in group definitions, and can be added to the project database 103 as project objects themselves. This allows for the project database 103 to constantly evolve to incorporate changes in project execution over time.

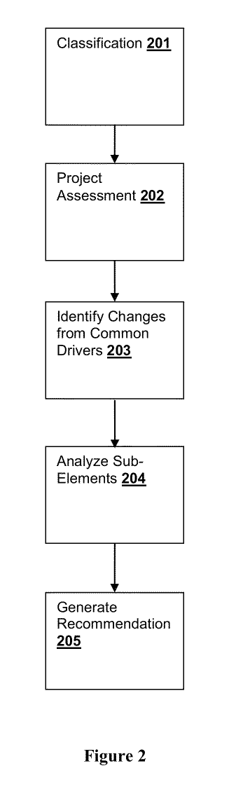

FIG. 2 illustrates an example execution of the classification and assessment of a project, as executed by the project recognition engine 101.

The process illustrated in FIG. 2 begins with a classification step 201, where each given project picture is classified into defined groups of projects. This classification can be independently undertaken utilizing two different but related multivariate statistical techniques. In embodiments, the classification techniques can be employed in parallel, where both techniques are run in parallel. The classification results can then be compared according to priority rules for the techniques, can be normalized for convergence, or can otherwise be used for error detection and correction. In embodiments, the classification can employ either technique.

In embodiments, the classification techniques can be employed in series, where one of the two techniques is first employed and the other of the two techniques can be used as necessary to verify the classification (e.g., if the classification using the first technique in series fails to properly classify the pictures, if the classification using the first technique fails to meet a confidence threshold, etc.).

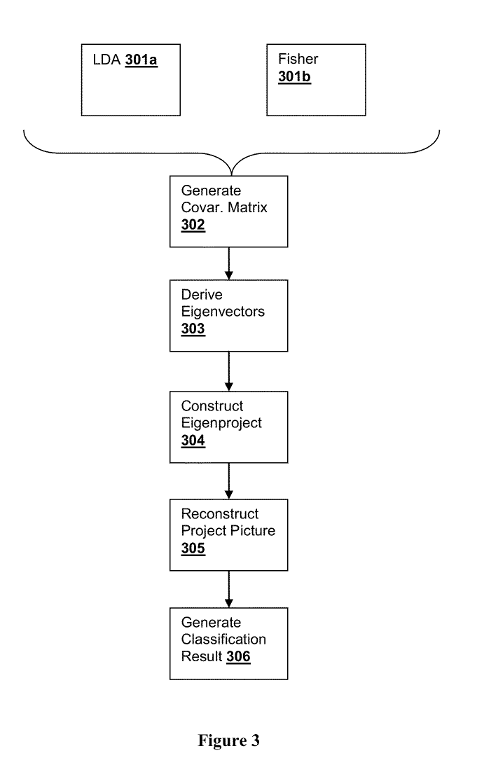

FIG. 3 provides a detailed illustrative view of an example classification step 201, employing the use of both multivariate statistical techniques in parallel.

The first multivariate statistical technique that can be utilized in classification is linear discriminant analysis (“LDA”), illustrated in step 301a. LDA is a statistical technique that can be employed in pattern recognition such as facial or voice recognition. LDA can be used to classify patterns based on a calculated Mahalanobis distance. In statistics, the Mahalanobis distance is a distance measure introduced by P. C. Mahalanobis in 1936. It is based on correlations between variables by which different patterns can be identified and analyzed. It gauges similarity of an unknown sample set to a known one. It differs from Euclidean distance in that it takes into account the correlations of the data set and is scale-invariant. In other words, it is a multivariate effect size.

An effect size calculated from data is a descriptive statistic can convey the estimated magnitude of a relationship without making any statement about whether the apparent relationship in the data reflects a true relationship in the population.

Each project picture being analyzed can be classified into the group whose mean is closest to it in the Mahalanobis sense.

LDA relies on key assumptions with respect to normal distribution of multivariate conditional probabilities and equivalence of group covariance matrices. These assumptions allow simplification of the analysis and can be useful in all but truly first of a kind projects where correlation with any defined group is weak at best. At step 302, the covariance matrix can be generated. In conducting LDA, a common group covariance matrix can be used, such as for a sufficiently robust common group. In embodiments, a pooled covariance matrix of all the groups in the project database can be used to strengthen the overall analysis using this type of pattern recognition.

For large groups, or for analysis using all groups in a database, the covariance matrix can be exceptionally large, and thus not be feasible to estimate. Techniques such as using principal component analysis (“PCA”) to reduce dimensionality by extracting so called principal components is equally infeasible for such a large covariance matrix.

The data matrix, however, can be recognized as being equal to the number of pixels times the number of pictures and the associated covariance matrix can be considered to consist of a number of non-zero eigenvectors equal to the number of project pictures in the data set. As such, a set of eigenvectors can be derived from the covariance matrix associated with the data matrix at step 303.

From this set of calculated eigenvectors, PCA can then be employed to construct one or more “eigenprojects” at step 304. In facial and speech recognition technologies, eigenvectors are used to calculate eigenfaces and eigenvoices, respectively.

Any given project picture can be reconstructed at step 305 by projecting it onto the eigenprojects with reconstruction complete when it has been projected using all the eigenprojects.

In an embodiment, project pictures can be projected onto a desired number of eigenprojects that is sufficiently large for analysis while remaining computationally cost-effective. For example, project pictures can be projected only onto approximately the first 20 eigenprojects and the new variables (principal component scores) used in LDA, such that the data matrix is equal to number of project pictures ×20 eigenprojects. Sensitivity tests of the training data sets can confirm the appropriateness of limiting projection to 20 eigenprojects by calculating the apparent error rate (“APER”), which can be considered an optimistic assessment of the actual error rate.

The second technique that can be utilized in classification is the Fisher discriminant method at step 301b. The Fisher discriminant method is similar in objective to PCA in the sense that it seeks to reduce dimensionality. However, the Fisher method does not make some of the assumptions of LDA, such as normally distributed classes or equal class covariances. Utilization of two dimensional Fisher discriminant space plots can be a useful tool to visualize the proximity of various groups in the classification system.

At step 306, the execution of the classification technique(s) used for classification results in a determined classification of the current project whose received project pictures were analyzed.

As pictures of a project are taken over time, the project represented by the project pictures can change or evolve. As these pictures are received, the classification process can be performed with each picture, or periodically over the life of a project. Over time it is possible for a project picture to indicate or suggest that the project should be otherwise categorized. This can result from one of two circumstances:

The first would be a significant enough change to the project picture over time such that it no longer ideally fits in the originally assigned group. Such reclassification can be the result of a determination that the project has different common descriptive features and attributes from the originally assigned group, and suggests changed areas of management focus and attention and new project areas of interest.

The second instance which can trigger project reclassification can be changes in the composite library of all project pictures such that groupings or the definitions of their characteristics changed as sample size grew.

If a project can be reclassified, a project manager or other user can be notified via the project interface 102 prior to the project assessment step 202, to allow for a decision on reclassification prior to subsequent assessment and analysis. Alternatively, the reclassification of a project can be presented to a project manager or other user as part of a recommendation, as described further below.

After classification, assessment step 202 is performed to identify areas of the current project that the project manager should focus on given the similarity of this project to some respective group of projects for which insight has been previously determined. In this example, insight can be considered to be observations and/or knowledge gained from the execution of the past project used in generating the project group of a specific type. Examples of insights can include causes, effects, conclusions, trends, project areas or parameters relevant to or associated with other insights, insight significance related to the project as a whole, etc. Insights for the initial eigenproject database (project object database) can come from one or more of a review of contemporaneously prepared project reports, lessons learned from previously executed projects of the type, reports prepared for previously executed projects, interviews with people having involvement with the project (e.g., project manager, executives, employees participating in the projects), market data for similar projects, etc.

The assessment 202 of initial database projects and other subsequent projects captured in the ever growing project picture database also enables an identification of areas of likely challenge, opportunity areas to explore, and can highlight important project factors affecting a project based on pattern relevant experience that would not otherwise readily evident. The assessment can be performed for a single project picture (e.g., the most current project picture of a current project), or can be performed for a plurality of project pictures (e.g., past project pictures, and can include the most current project picture). This can enable the assessment of areas of interest as described above for a current project's current state, as well as enabling an identification of trends that in turn allow for predictive analysis of a current project in a current state. Utilizing a pattern recognition type approach based on multivariate statistical techniques provides the project manager with an additional tool to manage the project.

At step 203, the project recognition engine 101 can identify subtle but pervasive changes to the project picture from potentially correlated common drivers. Examples of common drivers can include constraint-coupled factors and/or risks that are not readily apparent or easily observable, and systemic factors and/or risks having complex inter-relationships. These pervasive changes can be thought of as a “darkening” or “lightening” of the project picture, where the pervasive changes can collectively affect some or all of the pixels of a project picture. Early recognition of potential common drivers acting on the project provides an ability to seek out, understand and manage these drivers to the advantage of the project. In this case the comparative analysis can be between project pictures taken at different times. Both an absolute comparison (e.g., between the different project pictures of the current project taken at different times themselves) and comparison of respective eigenproject values from the database can be performed.

As described above, the analysis performed in steps 202 and 203 can be based on multiple project pictures taken over time. The project recognition engine 101 can use the aggregated project pictures over time to generate eigenproject “movies” for the current project. Likewise, eigenproject movies can be generated for a correlated group of project objects. The eigenproject “movies” of a current project can then be compared with a similar eigenproject movie for the correlated group, allowing a deeper understanding of how group values change with aging over a project's lifetime, and how those group values can ultimately affect the outcome of a completed project. This allows for the identification of trends for particular aspects of a project as well as for the project as a whole, and the interrelationship of different factors that affect a project's aging. Trends and aging tendencies can be used to predict outcomes of a current project based on current trends associated with current project characteristics as well as predict effects of potential changes to one or more aspects of the current project.

In an illustrative example, the capability to understand project aging patterns and predict their effects can have particular relevance in the operating and maintenance phase of the project. For example, understanding aging patterns as they pertain to operating phases allow for the understanding of a current operating state of a plant or installation, how decisions related to operations can affect the progress and development of a project and/or the operating state of the plant/installation. In a maintenance phase, maintenance needs can be identified and predicted so that maintenance plans can be implemented prior to reaching a critical status, or ultimately, project failure.

At step 204, the project recognition engine 101 can identify subtle but pervasive changes in sub-elements of a project. To do so, project values for a current project associated with a particular sub-element can be identified. In an embodiment, these project values can be additional project values retrieved by the project recognition engine 101, which are identified based on their relationships one or more of the received current project pixels. As such, project pictures for sub-elements can be created and analyzed. In an embodiment, one or more eigenprojects can be generated for the combination of sub-elements. In an embodiment, separate eigenproject pictures can be created according to each individual sub-element. This enables for the determination of meaningful conclusions about the sub-elements and their relationship to the project can be drawn. In particular, there might be relevance in evaluating complex stakeholder situations or assumption migration in complex, long duration projects. In an illustrative example, sub-elements of a project can include stakeholder management, assumption and productivity. In this example, the separate pictures corresponding to the sub-elements of stakeholder management, assumption and productivity can be considered a “triple bottom line” for a project. Depending on the nature of the current project, one or more sub-elements may or may not exist in the project or be relevant to the project. In other situations, a particular sub-element may be relevant to the project, but not relevant to a particular project at that particular point in time. As such, step 204 can be optional for certain project types or certain project states.

At step 205, the system 100 (such as via the project recognition engine 101) can generate one or more recommendations 112 based on the analysis of the identified project object(s) Ill corresponding to the current project attributes 110. In an embodiment, the recommendation functions can be performed by a recommendation engine that is a part of or is in communication with the recognition engine 101.

In an embodiment, a recommendation 112 can include a recommendation to alter one or more aspects of a current project so that the project attributes reflecting those aspects are changed in a desired manner, thereby guiding the project towards a desired state (i.e., get the project “back on track”). The recommendation 112 can be to alter project aspects or parameters such that the project attributes 110 change to converge with the attributes of applicable project objects 111, and so the project as a whole is driven to align with the project object. The recommendation 112 can also (or alternatively) suggest a modification to aspects of the current project such that the project attributes diverge from corresponding object attributes of project objects 111 and, consequently, that the project diverges from the identified project objects 111 (e.g., if the identified project objects correspond to project objects of failed projects or reflect a project having an undesired cost, outcome or other undesired factors).

In an embodiment, the recommendation 112 can include a recommendation to change the objective, goal or purpose of the current project based on the current state of the project relative to identified project objects. For example, suppose that the classification analysis of a current project picture based on the attributes of a current, active project identifies a project object or project object group corresponding to the predefined or stated original goal or purpose of the current project, whereby the identified project object reflects a desired or optimal state of the current project (e.g., where the current project “should be” at the state reflected by the received attributes). However, in this example, suppose that the assessment of the attributes of the current project with those of the project object show a great disparity between the state of the current project and that of the project object. As such, the corrections required to get the current project “back on track” to an acceptable level can be likely to incur a substantial cost (e.g., financial, resources, effort, manpower, logistical, etc.). In this case, other project objects 111 (and/or project groups) may be found to be a better fit for the current project in its current state. In this case, the project recognition engine 101 can identify one or more project objects 111 (and/or project groups) that are better suited to the current project in its current state than the previously identified project object 111 corresponding to a previously categorized, predefined or pre-stated purpose of the project. This can be considered a reclassification of the current project. In an embodiment, the reclassification step can be performed, and can be a re-execution of the classification functions described above. The reclassification for a current project picture can be performed if the deviation from the originally identified project object and/or project group exceeds a particular threshold. In an embodiment, the reclassification can be performed periodically during the lifetime of a current project, as over time a project may gradually deviate and evolve into a project better suited to a different classification. When a more suitable project object is identified (e.g. via reclassification), the recommendation 112 can include a recommendation to shift the goal or purpose of the current project to that of the newly identified, more suitable project object. The recommendation 112 can include modifications or changes to one or more of the attributes of the current project to better align with the object attributes of the newly-identified project object. Therefore, while modifications may still be required, the cost of applying any changes to align the current project to achieve necessary project objectives is reduced and waste of efforts and progress of the current project to that point is minimized.

In an embodiment, it is contemplated that functions and methods of the inventive subject matter can be implemented across a program or a larger project portfolio, which can have large amounts of unrelated project and/or large amounts of unrelated or uncorrelated variables. In these embodiments, training database would be required to be programmatic or portfolio-oriented in nature.

In an embodiment, it is contemplated that external factors can be incorporated into the database, to facilitate assessments of resiliency. For example, external factors can be incorporated as external attributes, whereby the external attributes are associated with conditions that are not directly related to or typically associated with a particular project or project type, but that can have a ‘one-time’ impact on the current project.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A project analysis system comprising.
a project interface configured to receive project attributes of a project;
a project database storing project objects, each project object representative of a known aspect of a known project and including object attributes; and
a project recognition engine communicatively coupled with the project interface and the project database, and configured to:
obtain the project attributes via the project interface;
identify at least one project object as being similar to the project by analyzing the project attributes of the project with respect to object attributes of project objects in the project database; and
configure an output device to present the identified at least one project object.

2. The system of claim 1, wherein the project recognition engine is further configured to classify the project into a project class as a function of the identified at least one project object.

3. The system of claim 2, wherein the project class comprises an eigenproject.

4. The system of claim 3, wherein the at least one project object represents an eigenproject.

5. The system of claim 4, wherein the eigenproject corresponds to a reference class project, the eigenproject comprising at least one object eigenvector generated based on the object attributes.

6. The system of claim 5, wherein the project recognition engine configured to identify at least one project object as being similar to the project object further comprises the project recognition configured to identify the at least one project object by utilizing pattern recognition to compare the project attributes and the at least one object eigenvector.

7. The system of claim 1, wherein the project attributes comprise a snap shot in time of the project.

8. The system of claim 1, wherein the at least one project object comprises a snap shot in time of the corresponding known project.

9. The system of claim 1, wherein the project attributes and object attributes adhere to a common project namespace.

10. The system of claim 1, wherein the project comprises a capital construction project.

11. The system of claim 1, wherein the project comprises at least one of the following: a financial project, an engineering project, a design project, a construction project, a construction management project, a software development project, a supply chain project, a maintenance project, and a movie project.

12. The system of claim 1, wherein the project attributes include at least one of the following: a perception of an objective, a resource metric, a stakeholder value, a project driver, a logistic, and a relationship.

13. The system of claim 1, further comprising a recommendation engine communicatively coupled with the recognition engine and configured to:
obtain the project attributes and the at least one project object; and
generate a recommendation based on the project attributes and the at least one project object, the recommendation comprising suggested actions to alter the project.

14. The system of claim 13, wherein the suggested actions attempt to align the project with the at least one project object.

15. The system of claim 13, wherein the suggested actions attempt to direct the project away from alignment with the at least one project object.

16. A project analysis system comprising.
a project interface configured to receive project attributes of a project;
a project database storing project objects, each project object representative of a known aspect of a known project and including object attributes; and
a project recognition engine communicatively coupled with the project interface and the project database, and configured to:
calculate at least one object eigenvector for each of the stored project objects based on the object attributes of each of the stored project objects;
generate an eigenproject comprising at least one object eigenvector corresponding to at least one of the stored project objects, wherein the eigenproject represents a reference class project;
obtain the project attributes via the project interface;
identify at least one project object as being similar to the project by applying a pattern recognition technique to compare at least one object eigenvector from the eigenproject and the obtained project attributes; and
configure an output device to present the at least one identified project object.

17. The system of claim 16, wherein the project recognition engine is further configured to classify the project into a project class as a function of the identified at least one project object.

18. The system of claim 16, wherein the project attributes comprise a snap shot in time of the project.

19. The system of claim 16, wherein the at least one project object comprises a snap shot in time of the corresponding known project.

20. The system of claim 16, wherein the project attributes and object attributes adhere to a common project namespace.

21. The system of claim 16, wherein the project comprises a capital construction project.

22. The system of claim 16, wherein the project comprises at least one of the following: a financial project, an engineering project, a design project, a construction project, a construction management project, a software development project, a supply chain project, a maintenance project, and a movie project.

23. The system of claim 16, wherein the project attributes include at least one of the following: a perception of an objective, a resource metric, a stakeholder value, a project driver, a logistic, and a relationship.

24. The system of claim 16, further comprising a recommendation engine communicatively coupled with the recognition engine and configured to:
obtain the project attributes and the at least one project object; and
generate a recommendation based on the project attributes and the at least one project object, the recommendation comprising suggested actions to alter the project.

25. The system of claim 24, wherein the suggested actions attempt to align the project with the at least one project object.

26. The system of claim 24, wherein the suggested actions attempt to direct the project away from alignment with the at least one project object.
